(19) United States
(12) Patent Application Publication
(10) Pub. No.: US 2025/0208121 A1
(43) Pub. Date: Jun. 26, 2025

(54) MAMMALIAN PER- AND POLYFLUORINATED-ALKYL COMPOUND (PFAS) BINDING PROTEINS AND USES THEREOF

(71) Applicant: Allonnia, LLC, Boston, MA (US)

(72) Inventors: Dayal Saran, Melrose, MA (US); Areen Banerjee, Arlington, MA (US)

(21) Appl. No.: 18/991,608

(22) Filed: Dec. 22, 2024

Related U.S. Application Data
(60) Provisional application No. 63/613,984, filed on Dec. 22, 2023.

(51) Int. Cl. G01N 33/53; G01N 21/78; G01N 33/18

(52) U.S. Cl. CPC G01N 33/5308; G01N 21/78; G01N 33/18

(57) ABSTRACT

Provided herein are mammalian proteins that bind per- and polyfluoroalkyl compounds (PFAS), detection systems comprising the mammalian proteins, and uses thereof for detecting PFAS.

### FIELD OF THE INVENTION

The invention relates to mammalian per- and polyfluoroalkyl compound (PFAS) binding protein compositions, detection systems containing the mammalian PFAS binding proteins, and uses thereof for detecting PFAS.

### REFERENCE TO THE SEQUENCE LISTING

This application contains a Sequence Listing that has been submitted in XML format via Patent Center and is hereby incorporated by reference in its entirety. The file, created on Dec. 21, 2024, is named SequenceListing-117103-0101.01USNP.xml, and is 16,500 bytes in size.

### BACKGROUND OF THE INVENTION

Per- and Polyfluoroalkyl compounds are a class of substances that have found use in industrial & commercial manufacturing over the last six decades. Some properties that have made them attractive to the automotive, healthcare, energy & storage industries are their chemical inertness, thermal resistance & protection from weather & abrasion. The USEPA PFAS Action Plan, released in February 2019, indicated that the agency would establish a revised drinking water maximum contaminant level (MCL) in line with recent studies that have been performed on the deleterious effects of PFAS on human health and the environment. Existing market estimates place the size of the environmental liability in the US at over $80 billion. In March 2023, The EPA announced a proposed national Primary Drinking Water Regulation (PDWR) for six PFAS compounds including perfluorooctanoic acid (PFOA), perfluorooctane sulfonic acid (PFOS), perfluorononanoic acid (PFNA), hexafluoropropylene oxide dimer acid (HFPO-DA, commonly known as GenX Chemicals), perfluorohexane sulfonic acid (PFHxS), and perfluorobutane sulfonic acid (PFBS). EPA anticipates finalizing the regulation by the end of 2023. Current solutions for PFAS remediation are focused on sequestration techniques via adsorption media and then transfer to a landfill or incineration facility.

A big gap in the remediation effort is the rapid detection of ultra-low concentrations of PFAS. The ideal scenario for field detection would be portable chip-based unit that can detect single digit ppt levels of PFAS compounds with minimum operator actions within 1.5 hours of sampling. Another important factor in this detection system would be to have specificity of binding to PFAS compounds and avoid detection of interfering moiety like octanoic acid.

The main challenges involved in the biosensor progress are (i) the efficient capturing of biorecognition signals and the transformation of these signals into electrochemical, electrical, optical, gravimetric, or acoustic signals (transduction process), (ii) enhancing transducer performance i.e., increasing sensitivity, shorter response time, reproducibility, and low detection limits even to detect individual molecules, and (iii) miniaturization of the biosensing devices using micro- and nano-fabrication technologies. In light of the challenges in biosensor progress, there is a need in the art for improved binding proteins for detecting PFAS.

### SUMMARY OF THE INVENTION

Provided herein is a composition comprising a mammalian per- and polyfluoroalkyl compound (PFAS) binding protein, which may be a PFAS binding receptor. The PFAS binding protein may bind to one or more PFAS compounds (that is, may have PFAS compound binding activity). The mammalian PFAS binding protein may comprise a mammalian protein selected from the group consisting of Nucleoside diphosphate kinase, mitochondrial (NME4); FAM110D; Tumor protein p63-regulated gene 1 protein (TPRG1); Voltage-gated potassium channel subunit beta-1 (KCNAB1); Protein CutA (CUTA); Ferroptosis suppressor protein 1 (AIFM2); Polymerase delta-interacting protein 3 (POLDIP3); Zinc-binding alcohol dehydrogenase domain-containing protein 2 (ZADH2); Ubiquitin carboxyl-terminal hydrolase 21 (USP21); Lysine-specific demethylase 4D (KDM4D); thyroglobulin; and peroxisome proliferator-activated receptor alpha (PPARA); sequence substantially identical thereto; and a fragment thereof. In one example, the mammalian PFAS binding protein is a human protein or a mammalian homolog thereof. The mammalian PFAS binding protein may comprise or consist of the sequence set forth in one of SEQ ID NOs: 1-12 or a sequence at least 80% identical thereto. In one example, the mammalian PFAS binding protein comprises or consists of the sequence set forth in one of SEQ ID NOs: 1-12.

The mammalian PFAS binding protein may comprise a molecule suitable for attaching the mammalian PFAS binding protein to a solid substrate. The attachment may be direct or indirect. The attaching may comprise the mammalian PFAS binding protein being functionalized on, embedded in, or immobilized on a solid substrate. The solid substrate may be a plate, a bead, or a capillary bed. The mammalian PFAS binding protein may be attached using standard binding chemistry. The standard binding chemistry may comprise amide bonds attaching the mammalian PFAS binding protein to the solid substrate. The solid substrate may be coated with streptavidin and the mammalian PFAS binding protein may be biotinylated. The standard binding chemistry may comprise a His-tag attaching the mammalian PFAS binding protein to the solid substrate.

The one or more PFAS compounds may comprise one or more straight chain perfluorinated carboxylic acid molecules comprising a chain of 4 to 10 carbons in length. The PFAS compounds may comprise one or more straight chain perfluorinated sulfonic acid molecules comprising a chain of 4 to 10 carbons in length. The PFAS compounds may comprise at least one of perfluorobutanoic acid (PFBA), perfluoropentanoic acid (PFPeA), perfluorohexanoic acid (PFHxA), perfluoroheptanoic acid (PFHpA), perfluorooctanoic acid (PFOA), perfluorononanoic acid (PFNA), perfluorodecanoic acid (PFDA), perfluorobutane sulfonate (PFBS), perfluoropentane sulfonic acid (PFPeS), perfluorohexanesulphonic acid (PFHxS), perfluoroheptanesulfonic acid (PfHpS), perfluorooctane sulfonic acid (PFOS), perfluorononanesulfonic acid (PFNS), and perfluorodecane sulfonic acid (PFDS). The PFAS compounds may comprise one or more of PFBA, PFPeA, PFHxA, PFHpA, PFOA, PFNA, and PFDA. The PFAS compounds may comprise PFOA, PFOS, PFHxS, PFBA, or a combination thereof. The PFAS compounds may comprise PFOA. The mammalian PFAS binding protein may be bound to at least one of the PFAS compounds.

Provided herein is a PFAS detection system comprising the composition. The mammalian PFAS binding protein may be in a solution or attached to the solid substrate. A cartridge may comprise the solid substrate. The solid substrate may comprise an electroactive surface. The solid substrate may comprise an optical waveguide surface. The optical waveguide surface may comprise a channel. The solid substrate may comprise a metal. The metal may comprise gold.

The solid substrate may comprise one or more capillary beds or a series thereof used in lateral flow assays or competitive lateral flow assays as described herein. The solid substrate may comprise at least one functional molecule that binds the PFAS binding protein. The PFAS binding protein may be the mammalian PFAS binding protein.

Provided herein is a method of detecting a PFAS compound in a sample. The method may comprise contacting the sample with the composition or the detection system under conditions that allow the mammalian PFAS binding protein to bind the PFAS compound, resulting in a mammalian PFAS binding protein-PFAS compound conjugate. The method may comprise detecting the level of the mammalian PFAS binding protein-PFAS compound conjugate. The level of the mammalian PFAS binding protein-PFAS compound conjugate may be indicative of the level of the PFAS compound in the sample. The detecting may be by ELISA, an electrochemical method, an interferometry method, Surface Plasmon Resonance (SPR) method, or a flow-based immunoassay which may be a lateral flow assay or a competitive lateral flow assay.

Provided herein is a method of detecting a PFAS compound in a sample. The method may comprise contacting the sample with the composition or the PFAS detection system. The PFAS binding protein may be bound to an electroactive surface to create a mammalian PFAS binding protein-bound base. The contacting may be under conditions that allow the mammalian PFAS binding protein to bind the PFAS compound to result in a mammalian PFAS binding protein-PFAS compound conjugate. The method may comprise measuring electrochemical signals with a transducer before and after binding of the PFAS compound to the mammalian PFAS binding protein-bound base and calculating a difference between the electrochemical signals. A difference between the electrochemical signals before and after binding of the PFAS compound to the mammalian PFAS binding protein-bound base may correlate to the amount of the PFAS compound in the sample.

Provided herein is a method of detecting a PFAS compound in a sample. The method may comprise contacting the sample with the composition or the PFAS detection system under conditions suitable for the PFAS compound to bind to the mammalian PFAS binding protein to generate a PFAS compound-mammalian FAS binding protein conjugate. The mammalian PFAS binding protein may be bound to a surface of an optical planar waveguide. The method may comprise exposing the waveguide comprising the PFAS compound-mammalian PFAS binding protein conjugate to a sensing beam of polarized light. The method may comprise optically combining the sensing beam with an adjacent reference beam of polarized light to generate an interference pattern. The interference pattern may be indicative of a change in a speed of the sensing beam. The degree of change in the speed of the sensing beam may be indicative of the amount of the PFAS compound in the sample.

Provided herein is a method of detecting a PFAS compound in a water sample. The method may comprise contacting the sample to the composition or the PFAS detection system.

The mammalian PFAS binding protein may be immobilized on the solid substrate and may be attached to a detectable label. The contacting may be under conditions suitable for the PFAS compound to bind to the mammalian PFAS binding protein to generate a PFAS compound-mammalian PFAS binding protein conjugate. The solid substrate may comprise a first end, a first middle portion, a second middle portion, and a second end. The solid substrate may allow liquid to migrate from the first end through the middle portions to the second end through capillary action. The first middle portion may comprise the mammalian PFAS binding protein and the second middle portion may comprise a second binding molecule immobilized on the solid substrate. The second binding molecule may bind to the PFAS compound-mammalian PFAS binding protein conjugate. The sample may be contacted to the first end of the solid substrate. The method may comprise allowing the PFAS compound-mammalian PFAS binding protein conjugate to migrate from the first end through first middle portion into the second middle portion. The method may comprise detecting the detectable label in the second middle portion. Detecting the detectable label may indicate the presence of the PFAS compound in the sample. The detectable signal may be colorimetric or fluorescent.

Provided herein is a method of detecting a PFAS compound in a water sample. The method may comprise contacting the sample to the composition or the detection system. The mammalian PFAS binding protein may be immobilized on the solid substrate and may be attached to a detectable label. The contacting may be under conditions suitable for the PFAS compound to bind to the mammalian PFAS binding protein to generate a PFAS compound-mammalian PFAS binding protein conjugate. The solid substrate may comprise a first end, a first middle portion, a second middle portion, and a second end. The solid substrate may allow liquid to migrate from the first end through the middle portions to the second end through capillary action. The first middle portion may comprise the mammalian PFAS binding protein and the second middle portion may comprise the PFAS binding compound immobilized on the solid substrate. The sample may be contacted to the first end of the solid substrate. The method may comprise allowing the PFAS compound-mammalian PFAS binding protein conjugate to migrate from the first end through the first middle portion into the second middle portion. The method may comprise detecting the detectable label in the second middle portion. The absence of the detectable label may indicate the presence of the PFAS compound in the sample. The detectable signal may be colorimetric or fluorescent.

In the methods, the limit of detection of the method may be no greater than a single digit parts per trillion. The sample may be a water sample. The water sample may be pre-treated, which may be accomplished by concentrating the PFAS compound before the contacting step. The pre-treatment may comprise removing inhibitors of the binding of the PFAS binding protein and the PFAS compound. The compounds may be concentrated using solid phase extraction (SPE), such as with an SPE cartridge. The PFAS compound may be extracted in an organic solvent. The organic solvent may be ethanol, and the SPE cartridge may comprise a C-18 silica matrix. The organic solvent may comprise ethanol and ammonium hydroxide, which may be at less than or equal to 1.5% concentration, and the SPE cartridge may comprise a weak anion exchange matrix. The organic solvent may comprise ethanol and formic acid, which may be at less than or equal to 2% concentration, and the SPE cartridge may comprise a weak hydrophobic matrix.

In the methods, the PFAS compound in the sample may comprise one or more straight chain perfluorinated carboxylic acid- or sulfonic acid molecules comprising a chain of 4 to 10 carbons in length. The PFAS in the sample may comprise one or more of perfluorobutanoic acid (PFBA), perfluoropentanoic acid (PFPeA), perfluorohexanoic acid (PFHxA), perfluoroheptanoic acid (PFHpA), perfluorooctanoic acid (PFOA), perfluorononanoic acid (PFNA), perfluorodecanoic acid (PFDA), perfluorobutane sulfonate (PFBS), perfluoropentane sulfonic acid (PFPeS), perfluorohexanesulphonic acid (PFHxS), perfluoroheptanesulfonic acid (PfHpS), perfluorooctane sulfonic acid (PFOS), perfluorononanesulfonic acid (PFNS), and perfluorodecane sulfonic acid (PFDS). The PFAS compound in the sample may comprise one or more of PFBA, PFPeA, PFHxA, PFHpA, PFOA, PFNA, and PFDA. The PFAS in the sample may comprise one or more of PFOA, PFOS, PFHxS, and PFBA. The PFAS in the sample may comprise PFOA.

### BRIEF DESCRIPTION OF THE DRAWINGS

FIGS. 1A-B show the results of Surface Plasmon Resonance (SPR) for binding of the human proteins thyroglobulin (TG) (FIG. 1A) and peroxisome proliferator-activated receptor alpha (PPARA) (FIG. 1B) to different concentrations of PFOA.

### DETAILED DESCRIPTION

The focal activity of interest for the subject disclosed herein is the ability to use a mammalian protein for detecting Perfluorooctanoic acid (PFOA) and other PFAS compounds.

PFAS bind to proteins and this interaction has been postulated to play a major role in bioaccumulation and toxicity. Previous research suggests that the half-lives of PFOS and PFOA are approximately 5.4 and 3.8 years in humans indicating the strong possibility of multiple human proteins that interact with PFAS compounds. PFAS compounds have a strong charged head that makes them vey ‘sticky.’ The predicted model for PFAS accumulation in animals is by binding to proteins like Serum Albumin etc. The primary mode of PFAS related human toxicity is through bioaccumulation in the body. The main organs where bioaccumulation is observed is Liver and kidneys. These organs are sieves that filter contaminants from the serum.

The inventors had the insight that proteins that are expressed in these organs have a high likelihood of having strong binding to PFAS compounds. They hypothesized that screening & identifying novel proteins with binding affinity to low concentration of PFAS compounds would allow for development of ultra-sensitive bio-based sensors against specific PFAS compounds. The initial efforts were around testing a human protein microarray against binding affinity to PFAS compounds. The proteins were exposed to varying concentrations of PFOA-Biotin conjugate (10 ppm-10 ppb). The resulting binding was analyzed by adding a secondary Streptavidin based detection system. The positive proteins were quantified by giving the signal a Z-score which signifies the intensity of the signal. Following the z-score generations, the proteins were ranked.

The inventors have identified mammalian proteins that exhibit surprising sensitivity to PFAS compounds that allow detection of PFAS compounds with reduced background signal from non-fluorinated compounds. The proteins can be adapted into systems for fast and easy-to-use field kit biosensor to detect PFAS compounds at contaminated sites. Such applications will help users draw PFAS plume maps without having to spend valuable resources and time in sending samples to the lab for sensitive testing. Systems implementing the mammalian binding proteins will reduce the number of samples that have to be sent out for expensive laboratory testing and reduce the amount of time it takes to map the contaminated regions.

### 1. Definitions

Before the present compositions and methods are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

Unless defined otherwise, all technical and scientific terms used herein have the same meanings as commonly understood by one of ordinary skill in the art to which this invention belongs.

In describing and claiming the present invention, the following terminology will be used in accordance with the definitions set out below.

The terminology used herein is for the purpose of describing particular embodiments only and is not intended to be limiting. As used in the specification and the appended claims, the singular forms “a,” “an” and “the” include plural referents unless the context clearly dictates otherwise. Thus, for example, reference to a kit containing “a monoclonal antibody” includes a mixture of two or more monoclonal antibodies, reference to “an antibody” includes reference to two or more of such antibodies, and reference to “a PFAS” includes reference to a mixture of two or more PFAS.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, “comprising,” “including,” “containing,” “characterized by,” and grammatical equivalents thereof are inclusive or open-ended terms that do not exclude additional, unrecited elements or method steps.

A “peptide” or “polypeptide” or “protein” is a linked sequence of amino acids and may be natural, synthetic, or a modification or combination of natural and synthetic.

As used herein, “PFAS” means per- or polyfluoroalkyl compounds, “PFOA” means perfluorooctanoic acid; “PFOS” means perfluorooctanesulfonic acid, “PFHxS” means perfluorohexanesulfonic acid, “PFBA” means perfluorobutanoic acid, “PFPeA” means perfluoropentanoic Acid, “PFHxA” means perfluorohexanoic acid, “PFHpA” means perfluoroheptanoic acid, “PFNA” means, perfluorononanoic acid, “PFDA” means perfluorodecanoic acid, and “OA” means octanoic acid. As used herein, a “biosimilar” is an antibody that is highly similar to a reference antibody in both molecular structure and bioactivity, but bioactivity may differ from the reference antibody in some way that does not substantially affect bioactivity.

“Substantially identical” may mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 amino acids.

A “variant” may mean a peptide or polypeptide or protein that differs in amino acid sequence by the insertion, deletion, or conservative substitution of amino acids, but retains at least one biological activity. Representative examples of “biological activity” include the ability to bind to a toll-like receptor and to be bound by a specific antibody. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

### 2. PFAS Binding Proteins

Provided herein is a mammalian PFAS binding protein, which may be a PFAS binding receptor. The mammalian PFAS binding protein may bind to one or more PFAS compounds. The mammal may be a mouse, rabbit, rat, dog, pig, non-human primate, or human. In one example, the mammal is a human. The PFAS compounds may comprise a straight chain PFAS compound.

The PFAS compounds may comprise one or more straight chain perfluorinated carboxylic- or sulfonic acid molecules of 4 to 10 carbons in length. The PFAS compounds may comprise one or more of perfluorobutanoic acid (PFBA), perfluoropentanoic acid (PFPeA), perfluorohexanoic acid (PFHxA), perfluoroheptanoic acid (PFHpA), perfluorooctanoic acid (PFOA), perfluorononanoic acid (PFNA), and perfluorodecanoic acid (PFDA). The PFAS compounds may also comprise one or more of perfluorobutane sulfonate (PFBS), perfluoropentane sulfonic acid (PFPeS), perfluorohexanesulphonic acid (PFHxS), perfluoroheptanesulfonic acid (PfHpS), perfluorooctane sulfonic acid (PFOS), perfluorononanesulfonic acid (PFNS), and perfluorodecane sulfonic acid (PFDS). In one example, the PFAS compounds comprise one or more of PFOA, PFOS, PFHxS, and PFBA. In another example, the PFAS compounds comprise PFOA.

The mammalian PFAS binding protein may comprise a human protein or a mammalian homolog thereof, which may be Nucleoside diphosphate kinase, mitochondrial (NME4); FAM110D; Tumor protein p63-regulated gene 1 protein (TPRG1); Voltage-gated potassium channel subunit beta-1 (KCNAB1 or KCAB1); Protein CutA (CUTA); Ferroptosis suppressor protein 1 (AIFM2); Polymerase delta-interacting protein 3 (POLDIP3); Zinc-binding alcohol dehydrogenase domain-containing protein 2 (ZADH2 or Prostaglandin reductase 3 or PTGR3); Ubiquitin carboxyl-terminal hydrolase 21 (USP21); Lysine-specific demethylase 4D (KDM4D); thyroglobulin; or peroxisome proliferator-activated receptor alpha (PPARA). The human protein may comprise the sequence set forth, respectively, in SEQ ID NOs: 1-12.

The human PFAS binding protein may comprise a sequence substantially identical to one of SEQ ID NOs: 1-12. In one example, the human PFAS binding protein comprises a sequence of at least 70, 75, 80, 85, 90, 91, 92, 93, 94, 95, 96, 97, 98, or 99% identical to one of SEQ ID NOs: 1-12. The human PFAS binding protein may comprise a fragment of one of the foregoing human proteins or substantially identical sequences thereto. In one example, the fragment comprises at least 10, 20, 30, 40, 50, 60, 70, 80, 90, 100, 110, 120, 130, 140, 150, 160, 170, 180, 190, or 200 amino acids of the human protein, one of SEQ ID NO: 1-12, or the sequence substantially identical thereto.

The mammalian PFAS binding protein may be recombinant. The recombinant protein may be expressed in bacterial or eukaryotic cells.

The mammalian PFAS binding protein may comprise a molecule suitable for attaching the mammalian PFAS binding protein to a solid substrate. The molecule may comprise biotin, the molecule may mammalian a His-tag.

The mammalian PFAS binding protein may be in a solution. The solution may comprise physiological saline, which may be buffered with potassium phosphate.

### 3. PFAS DETECTION SYSTEMS AND METHODS OF DETECTING PFAS

Provided herein is a PFAS detection system comprising one or more mammalian PFAS binding proteins disclosed herein. Also provided herein are methods of detecting the one or more PFAS compounds from a sample containing or suspected of containing one or more PFAS compounds. The methods may be carried out using the PFAS detection system. The detection system may comprise a solution comprising the mammalian PFAS binding protein or a solid substrate comprising the mammalian PFAS binding protein. This detection system and methods may detect at least 4 ppt, and, preferably, single digit ppt of PFAS compounds, and may increase specificity of a detection method towards PFAS compounds.

In one example, the PFAS compounds are concentrated before being applied to the PFAS detection system. The compounds may be concentrated using solid phase extraction (SPE), such as with an SPE cartridge. The PFAS compounds may be extracted in an organic solvent. The organic solvent may be ethanol, and the SPE cartridge may comprise a C-18 silica matrix. The organic solvent may comprise ethanol and ammonium hydroxide, which may be at less than or equal to 1.5% concentration, and the SPE cartridge may comprise a weak anion exchange matrix. The organic solvent may comprise ethanol and formic acid, which may be at less than or equal to 2% concentration, and the SPE cartridge may comprise a weak hydrophobic matrix. A sample comprising the PFAS compounds may be treated to degrade potential inhibitors.

The mammalian PFAS binding protein may be immobilized on the solid substrate, and the immobilization may comprise a covalent or non-covalent attachment. The solid substrate may comprise a coating capable of binding the human PFAS binding protein. The mammalian PFAS binding protein may be attached to the solid substrate using standard binding chemistry. In one example, the PFAS binding protein is attached using amide bonds. In another example, the mammalian PFAS binding protein is attached to the solid substrate using a His-tag. The substrate may comprise an electroactive surface, which may comprise glass. The mammalian PFAS binding protein may be bound to the coating or electroactive surface. In one example, the coating comprises streptavidin, NeutrAvidin, or a biotin-binding protein, and the mammalian PFAS binding protein is biotinylated. In another example, the solid substrate comprises an anti-mammalian PFAS binding protein antibody and the mammalian PFAS binding protein is bound to the mammalian PFAS binding protein antibody.

The solid substrate may be a bead or a plate, or any other suitable material for use in protein-based detection assays. The solid substrate may comprise an electroactive surface. The solid substrate may comprise a strip, which may be suitable for use in flow-based assays such as lateral flow and competitive lateral flow assays. The solid substrate may comprise at least one functional molecule that binds the PFAS binding protein. A cartridge may comprise the solid substrate. The mammalian PFAS binding protein or the solid substrate may be deployed in a biosensor system to detect the one or more PFAS compounds.

The detection system may be a flow-based detection system, which may be a lateral flow (LF) detection system or a competitive lateral flow (CLF) detection system. The flow-based detection system may comprise the mammalian PFAS binding protein, which may be conjugated to a bead comprising a dye, a metal bead, or fluorescent active molecules or electroactive ions that can produce electrochemiluminescence, which may be functionalized on a solid substrate. The mammalian PFAS binding protein may be detectably labeled and may produce a detectable signal. The detectable label may be directly or indirectly attached to the PFAS binding protein. The solid substrate may allow fluid migration through capillary action. The solid substrate may comprise a first end, a first middle portion, a second middle portion, and a second end. The fluid may migrate from the first end through the middle portions to the second end. The mammalian PFAS binding protein may be immobilized in the first middle portion.

The solid substrate may comprise one or more capillary beds. The capillary beds may be arranged as a series such that liquid can flow across the series. Each capillary bed in the series may in sequence comprise the first end, the first middle portion, the second middle portion, and the second end. The first end may be suitable for introducing a sample to the detection system. The first middle portion may comprise the mammalian binding protein and optionally reagents for detecting the PFAS compound. The second end may be suitable for absorbing excess sample fluid and waste fluid. The capillary beds may comprise pieces of porous paper, micro-structured polymer, or sintered polymer. The solid substrate may be a strip.

The LF detection system may comprise the mammalian PFAS binding protein immobilized on the solid substrate in the first middle portion. The LF detection system may comprise a second binding molecule that recognizes a different moiety of the PFAS compound as compared to the mammalian PFAS binding protein or the PFAS compound-mammalian PFAS binding protein conjugate. The second binding molecule may be immobilized on the same solid substrate as the PFAS binding protein, but at a distance from the PFAS binding protein, which may be in the second middle portion. The second binding molecule may be a polymer. The polymer may be an organic molecule. The polymer may be an inorganic molecule.

The PFAS binding protein may trap the PFAS compound from a sample as it migrates across the solid substrate. The second binding molecule may trap the PFAS binding protein bound to the PFAS compound or the PFAS compound-mammalian PFAS binding protein conjugate. The second binding molecule may be arranged downstream of the PFAS binding protein, in the direction of fluid flow across the solid substrate. The second binding molecule may be arranged in a line perpendicular to the direction of fluid flow, but other arrangements are possible. Binding of the second binding molecule to the PFAS binding protein-PFAS compound conjugate may result in a detectable signal indicative of the presence of the PFAS compound in the sample. Binding of the second molecule to the PFAS compound-mammalian PFAS binding protein conjugate may concentrate the amount of signal produced by the mammalian binding protein, thereby generating a detectable signal.

The CLF detection system may comprise the PFAS compound immobilized on the solid substrate, which may be in the first middle portion. The CLF detection system may lack a second middle portion. The PFAS binding protein may trap the PFAS compound from a sample, and the PFAS binding protein-PFAS compound complex may not be trapped by the immobilized PFAS compound. The presence of the PFAS compound in the sample may result in the lack of a detectable signal from the CLF detection system. The absence of, or the presence of the PFAS below a detection threshold, in the sample may result in a detectable signal from the CLF detection system.

The flow-based detection system may comprise a detectable signal that is colorimetric or fluorescent. The detectable label may produce a visible, fluorescent, or electrochemiluminescent signal. The flow-based detection system may comprise an electroactive dye to amplify a luminescent signal. The detectable label may comprise an electroactive label, which may be a ferrocene derivative. The electroactive label may be ferrocenecarboxylic acid, anthraquion-one 2-carboxylic acid, thionine, tris (2,2′-bipyridine-4,4′-dicarboxylic acid)cobalt(III), tris(bipyridine)ruthenium(II) with an N-succinimidyl ester group, or an iron heme group in horseradish peroxidase. The electroactive label may be phenazine dye, neutral red, toluidine blue, Prussian blue, methylene blue, azure A, thionine, anthraquinone, or tris(bipyridine)ruthenium (II) [Ru(bpy)₃]²⁺.

In another example, the detection system may comprise the human PFAS binding protein and a biotin-conjugated analyte. A sample suspected of containing PFAS may be contacted to the human PFAS binding protein. The PFAS and biotin-conjugated analyte may compete for binding, and an enzymatic detection molecule that binds the biotin-conjugated analyte may be added. An enzyme substrate may be added, and an enzymatic color reaction may proceed. The amount of color may be proportional to the amount of bound conjugate.

A biotin-conjugated analyte and a sample suspected of containing the one or more PFAS compounds may be contacted to the human PFAS binding protein. The one or more PFAS compounds and biotin-conjugated analyte may compete for binding, and an enzymatic detection molecule that binds the biotin-conjugated analyte may be added. An enzymatic color reaction may occur, and the amount of color may be proportional to the amount of bound conjugate.

A biotin-conjugated analyte may be contacted to a surface coated with streptavidin and captured by biotin-streptavidin binding. The human PFAS binding protein and a sample suspected of containing the one or more PFAS compounds may be introduced to the cartridge, and the one or more PFAS compounds may compete for binding with the human PFAS binding protein. An anti-human PFAS binding protein conjugated with horseradish peroxidase (HRP) may be added, and an enzymatic color reaction may occur. The amount of color may be proportional to the amount of bound conjugate. In another example, an anti-human PFAS binding protein antibody may be added, an anti-IgG antibody conjugated with HRP may be added, and an enzymatic color reaction may occur. The amount of color may be proportional to the amount of bound conjugate.

The system may comprise the human PFAS binding protein bound to an electroactive surface, which may create a baseline electrochemical property. A water sample suspected of containing the one or more PFAS compounds may be passed over the surface, where the one or more PFAS compounds may preferentially bind to the human PFAS binding protein. The binding may alter the electrochemical properties of the surface. A voltametric reading using a potentiostat may deliver an altered signal, which may be measured by a transducer and displayed on a signal display readout. The amount of PFAS binding events may be proportional to the altered electrochemical signal measured. The human PFAS binding protein may comprise an electroactive label conjugate. The electroactive label may be a ferrocene derivative. The electroactive label may be ferrocenecarboxylic acid, anthraquion-one 2-carboxylic acid, thionine, tris (2,2′-bipyridine-4,4′-dicarboxylic acid)cobalt(III), tris(bipyridine)ruthenium(II) with an N-succinimidyl ester group, or an iron heme group in horseradish peroxidase. The electroactive label may be phenazine dye, neutral red, toluidine blue, Prussian blue, methylene blue, azure A, thionine, anthraquinone, or tris(bipyridine)ruthenium (II) [Ru(bpy)₃]²⁺. The human PFAS binding protein may be covalently bound to the electroactive surface. In one example, the electroactive surface comprises a gold surface. The gold surface made be modified with 3,3′-dithiobis (sulfosuccinimidyl) propionate (DTSSP). In another example, the human PFAS binding protein is immobilized and is thiolated.

The system may combine two very sensitive methods, waveguiding and interferometry, to form waveguide interferometry technology for use in rapid low-level detection sensing applications. The mammalian PFAS binding protein may be coupled to the waveguide surface. As the one or more PFAS compounds are introduced to the functionalized waveguide, the binding of PFAS to the mammalian PFAS binding protein may displace a sample solution near the waveguide surface, changing the light beam's velocity; an adjacent reference beam may be left unperturbed and optically combined with the sensing beam to measure the velocity change. This may create an interference pattern that shifts as the refractive index changes, producing a corresponding change in the relative phase measurement.

The detection may be based on an interferometry method, which may be surface plasmon resonance (SPR). In one example, the detection system comprises the PFAS binding protein bound to a metal surface, which may be a sheet. The metal may be functionalized to bind proteins. The metal may become excited by light at a specific angle of incidence. The metal sheet may be thin enough such that a surface plasmon resonance signal is triggered by a refractive index of the PFAS binding protein. Binding of the PFAS binding protein to the PFAS compound may cause a change in the refractive index. The degree of change may be proportional to the amount of PFAS compound in the sample. In another example, a device disclosed herein may be used to detect the one or more PFAS compounds using waveguide interferometry. The device may comprise a waveguide, which may be functionalized to bind proteins. The human PFAS binding protein may be bound to the waveguide.

The stability of the human PFAS binding protein and its response to inhibitory components in a “real world” matrix may be tested. After addressing these variables, various scenarios of field kit development may be tested. Components of the detection system may be provided in a kit. The kit may comprise one or more of the mammalian PFAS binding protein in solution, the solid substrate bound to the mammalian PFAS binding protein, a conjugated analyte, an enzymatic detection molecule, an anti-mammalian PFAS binding protein antibody optionally bound to an enzymatic detection molecule, an anti-IgG antibody optionally bound to an enzymatic detection molecule, an enzyme substrate, and one or more negative or positive controls. The conjugated analyte may be a biotin-conjugate analyte.

In one example, the kit may comprise the human PFAS binding protein immobilized directly on electrodes (which may be termed an immunosensor). The electrodes may be contained in a flow cartridge. A liquid sample, such as groundwater, suspected of containing the one or more PFAS compounds may be added to facilitate washing, incubation, and measurement steps. A field kit comprising the immunosensor may be weather resistant; may be battery powered; may have GPS capabilities; or may have capacity to measure four samples simultaneously; or a combination of the foregoing. Upon completion of the incubation and washing, a software program may measure the amount of the one or more PFAS compounds in the sample and produce results that can be visualized or exported in a spreadsheet format.

The field kit may be used for detection of the one or more PFAS compounds at a contaminated site. The field kit may be used to draw PFAS plume maps without having to spend valuable resources and time in sending samples to a laboratory for sensitive testing. A method of detecting the one or more PFAS compounds disclosed herein, if combined with a field kit, may reduce the number of samples that have to be sent out for expensive laboratory testing and reduce the amount of time it takes to map contaminated sites.

The present invention has multiple aspects, illustrated by the following non-limiting examples.

### Example 1

### Human PFAS Binding Proteins

Binding of perfluorooctanoic acid (PFOA) and other PFAS compounds to proteins has been postulated to play a major role in bioaccumulation and toxicity. Previous research suggests that the half-lives of PFOS and PFOA are approximately 5.4 and 3.8 years in humans, indicating the strong possibility of multiple human proteins that interact with PFAS compounds. PFAS compounds have a strong charged head that makes them very “sticky.” The predicted model for PFAS accumulation in animals is by binding to proteins like Serum Albumin (4). The primary mode of PFAS related human toxicity is through bioaccumulation in the body. The main organs where bioaccumulation is observed in liver and kidneys. These organs are sieves that filter contaminants from the serum. We hypothesized that proteins that are expressed in these organs have a high likelihood of having strong binding to PFAS compounds. It is also hypothesized that screening and identifying novel proteins with binding affinity to low concentration of PFAS compounds will allow for development of ultra-sensitive bio-based sensors against specific PFAS compounds.

The initial efforts were around testing a human protein microarray against binding affinity to PFAS compounds. The proteins were exposed to varying concentrations of PFOA-biotin conjugate (10 ppm-10 ppb). The resulting binding was analyzed by adding a secondary streptavidin-based detection system. The positive proteins were quantified by giving the signal a Z-score which signifies the intensity of the signal. It is calculated by the following equation.

Z=[F635−F635(avg)]/F635(std)

where F635(avg) and F635(std) are the average and standard deviation of the F635 values of all spots on the array, respectively. F635 is the average foreground signal intensity of 2 replicate spots of a given protein in the detection channel (635 nm) for human IgG. After generating these values, we ranked the positive protein lists (from all concentration of binding to PFOA) by using the following rules:
1. Took the average rank of each protein across all 4 dilutions (stored in the “average rank” column), with the rank based only on its Z-score.
2. Ranked all proteins based on their average rank.
3. Found the standard deviation of the average rank as a measure of the amount of variation in its rank across all 4 datasets. Therefore, the proteins with a lower standard deviation were more consistent at their average rank values across all dilutions.

This analysis weighs all of the dilutions equally. We identified the top 10 performers for binding to PFOA, which are provided in Table 1.

TABLE 1

| Protein Name | Amino Acid Sequence | SEQ ID NO: |
|---|---|---|
| NME4 | MGGLFWRSALRGLRCGPRAPGPSLLVRHGSGGPSWTRERTLVAVKPDGVQRRLVGD | 1 |
| | VIQRFERRGFTLVGMKMLQAPESVLAEHYQDLRRKPFYPALIRYMSSGPVVAMVWE | |
| | GYNVVRASRAMIGHTDSAEAAPGTIRGDFSVHISRNVIHASDSVEGAQREIQLWFQ | |
| | SSELVSWADGGQHSSIHPA | |
| FAM110D | MLLAPPSTPSRGRTPSAVERLEADKAKYVKTHQVIARRQEPALRGSPGPLTPHPCN | 2 |
| | ELGPPASPRTPRPVRRGSGRRLPRPDSLIFYRQKRDCKASVNKENAKGQGLVRRLF | |
| | LGAPRDAAPSSPASTERPAASGGWAAPQDAPEAAGKRALCPTCSLPLSEKERFFNY | |
| | CGLERALVEVLGAERFSPQSWGADASPQAGTSPPPGSGDASDWTSSDRGVDSPGGA | |
| | GGGGGSEAAGSARDRRPPVSVVERNARVIQWLYGCQRARGPPRESEV | |
| TPRG1 | MSTIGSFEGFQAVSLKQEGDDQPSETDHLSMEEEDPMPRQISRQSSVTESTLYPNP | 3 |
| | YHQPYISRKYFATRPGAIETAMEDLKGHVAETSGETIQGFWLLTKIDHWNNEKERI | |
| | LLVTDKTLLICKYDFIMLSCVQLQRIPLSAVYRICLGKFTFPGMSLDKRQGEGLRI | |
| | YWGSPEEQSLLSRWNPWSTEVPYATFTEHPMKYTSEKFLEICKLSGFMSKLVPAIQ | |
| | NAHKNSTGSGRGKKLMVLTEPILIETYTGLMSFIGNRNKLGYSLARGSIGF | |
| KCNAB1 | MLAARTGAAGSQISEENTKLRRQSGFSVAGKDKSPKKASENAKDSSLSPSGESQLR | 4 |
| | ARQLALLREVEMNWYLKLCDLSSEHTTVCTTGMPHRNLGKSGLRVSCLGLGTWVTF | |
| | GGQISDEVAERLMTIAYESGVNLEDTAEVYAAGKAEVILGSIIKKKGWRRSSLVIT | |
| | TKLYWGGKAETERGLSRKHIIEGLKGSLQRLQLEYVDVVFANRPDSNTPMEEIVRA | |
| | MTHVINQGMAMYWGTSRWSAMEIMEAYSVARQFNMIPPVCEQAEYHLFQREKVEVQ | |
| | LPELYHKIGVGAMTWSPLACGIISGKYGNGVPESSRASLKCYQWLKERIVSEEGRK | |
| | QQNKLKDLSPIAERLGCTLPQLAVAWCLRNEGVSSVLLGSSTPEQLIENLGAIQVL | |
| | PKMTSHVVNEIDNILRNKPYSKKDYRS | |
| CUTA | MSGGRAPAVLLGGVASLLLSFVWMPALLPVASRLLLLPRVLLTMASGSPPTQPSPA | 5 |
| | SDSGSGYVPGSVSAAFVTCPNEKVAKEIARAVVEKRLAACVNLIPQITSIYEWKGK | |
| | IEEDSEVLMMIKTQSSLVPALTDFVRSVHPYEVAEVIALPVEQGNFPYLQWVRQVT | |
| | ESVSDSITVLP | |
| AIFM2 | MGSQVSVESGALHVVIVGGGFGGIAAASQLQALNVPFMLVDMKDSFHHNVAALRAS | 6 |
| | VETGFAKKTFISYSVTFKDNFRQGLVVGIDLKNQMVLLQGGEALPFSHLILATGST | |
| | GPFPGKFNEVSSQQAAIQAYEDMVRQVQRSRFIVVVGGGSAGVEMAAEIKTEYPEK | |
| | EVTLIHSQVALADKELLPSVRQEVKEILLRKGVQLLLSERVSNLEELPLNEYREYI | |
| | KVQTDKGTEVATNLVILCTGIKINSSAYRKAFESRLASSGALRVNEHLQVEGHSNV | |
| | YAIGDCADVRTPKMAYLAGLHANIAVANIVNSVKQRPLQAYKPGALTFLLSMGRND | |
| | GVGQISGFYVGRLMVRLTKSRDLFVSTSWKTMRQSPP | |
| POLDIP3 | MADISLDELIRKRGAAAKGRLNARPGVGGVRSRVGIQQGLLSQSTRTATFQQRFDA | 7 |
| | RQKIGLSDARLKLGVKDAREKLLQKDARFRIKGKVQDAREMLNSRKQQTTVPQKPR | |
| | QVADAREKISLKRSSPAAFINPPIGTVTPALKLTKTIQVPQQKAMAPLHPHPAGMR | |
| | INVVNNHQAKQNLYDLDEDDDGIASVPTKQMKFAASGGFLHHMAGLSSSKLSMSKA | |
| | LPLTKVVQNDAYTAPALPSSIRTKALTNMSRTLVNKEEPPKELPAAEPVLSPLEGT | |
| | KMTVNNLHPRVTEEDIVELFCVCGALKRARLVHPGVAEVVFVKKDDAITAYKKYNN | |
| | RCLDGQPMKCNLHMNGNVITSDQPILLRLSDSPSMKKESELPRRVNSASSSNPPAE | |
| | VDPDTILKALFKSSGASVTTQPTEFKIKL | |
| ZADH2 | MLRLVPTGARAIVDMSYARHFLDFQGSAIPQAMQKLVVTRLSPNFREAVTLSRDCP | 8 |
| | VPLPGDGDLLVRNRFVGVNASDINYSAGRYDPSVKPPFDIGFEGIGEVVALGLSAS | |
| | ARYTVGQAVAYMAPGSFAEYTVVPASIATPVPSVKPEYLTLLVSGTTAYISLKELG | |
| | GLSEGKKVLVTAAAGGTGQFAMQLSKKAKCHVIGTCSSDEKSAFLKSLGCDRPINY | |
| | KTEPVGTVLKQEYPEGVDVVYESVGGAMEDLAVDALATKGRLIVIGFISGYQTPTG | |
| | LSPVKAGTLPAKLLKKSASVQGFFLNHYLSKYQAAMSHLLEMCVSGDLVCEVDLGD | 9 |
| | LSPEGRFTGLESIFRAVNYMYMGKNTGKIVVELPHSVNSKL | |
| USP21 | MPQASEHRLGRTREPPVNIQPRVGSKLPFAPRARSKERRNPASGPNPMLRPLPPRP | |
| | GLPDERLKKLELGRGRTSGPRPRGPLRADHGVPLPGSPPPTVALPLPSRTNLARSK | |
| | SVSSGDLRPMGIALGGHRGTGELGAALSRLALRPEPPTLRRSTSLRRLGGFPGPPT | |
| | LFSIRTEPPASHGSFHMISARSSEPFYSDDKMAHHTLLLGSGHVGLRNLGNTCFLN | |
| | AVLQCLSSTRPLRDFCLRRDFRQEVPGGGRAQELTEAFADVIGALWHPDSCEAVNP | |
| | TRFRAVFQKYVPSFSGYSQQDAQEFLKLLMERLHLEINRRGRRAPPILANGPVPSP | |
| | PRRGGALLEEPELSDDDRANLMWKRYLEREDSKIVDLFVGQLKSCLKCQACGYRST | |
| | TFEVFCDLSLPIPKKGFAGGKVSLRDCFNLFTKEEELESENAPVCDRCRQKTRSTK | |
| | KLTVQRFPRILVLHLNRFSASRGSIKKSSVGVDFPLQRLSLGDFASDKAGSPVYQL | |
| | YALCNHSGSVHYGHYTALCRCQTGWHVYNDSRVSPVSENQVASSEGYVLFYQLMQE | |
| | PPRCL | |
| KDM4D | METMKSKANCAQNPNCNIMIFHPTKEEFNDEDKYIAYMESQGAHRAGLAKIIPPKE | 10 |
| | WKARETYDNISEILIATPLQQVASGRAGVFTQYHKKKKAMTVGEYRHLANSKKYQT | |
| | PPHQNFEDLERKYWKNRIYNSPIYGADISGSLFDENTKQWNLGHLGTIQDLLEKEC | |
| | GVVIEGVNTPYLYFGMWKTTFAWHTEDMDLYSINYLHLGEPKTWYVVPPEHGQRLE | |
| | RLARELFPGSSRGCGAFLRHKVALISPTVLKENGIPFNRITQEAGEFMVTFPYGYH | |
| | AGFNHGENCAEAINFATPRWIDYGKMASQCSCGEARVTFSMDAFVRILQPERYDLW | |
| | KRGQDRAVVDHMEPRVPASQELSTQKEVQLPRRAALGLRQLPSHWARHSPWPMAAR | |
| | SGTRCHTLVCSSLPRRSAVSGTATQPRAAAVHSSKKPSSTPSSTPGPSAQIIHPSN | |
| | GRRGRGRPPQKLRAQELTLQTPAKRPLLAGTTCTASGPEPEPLPEDGALMDKPVPL | |
| | SPGLQHPVKASGCSWAPVP | |

We also performed Surface Plasmon Resonance (SPR) for a group of human proteins and studied their binding characteristics to PFOA. We identified two human proteins that showed promising binding properties against PFOA. The two proteins are). Their sequences are shown in Table 2.

TABLE 2

| Protein Name | Symbol | Amino Acid Sequence | SEQ ID NO: |
|---|---|---|---|
| Thyroglobulin | tg | IPRKPISKRPVRPSLPRSPRCPLPFNASEVVGGTILCET | 11 |
| | | ISGPTGSAMQQCQLLCRQGSWSVFPPGPLICSLESGRWE | |
| | | SQLPQPRACQRPQLWQTIQTQGHFQLQLPPGKMCSADYA | |
| | | GLLQTFQVFILDELTARGFCQIQVKTFGTLVSIPVCNNS | |
| | | SVQVGCLTRERLGVNVTWKSRLEDIPVASLPDLHDIERA | |
| | | LVGKDLLGRFTDLIQSGSFQLHLDSKTFPAETIRFLQGD | |
| | | HFGTSPRTWFGCSEGFYQVLTSEASQDGLGCVKCPEGSY | |
| | | SQDEECIPCPVGFYQEQAGSLACVPCPVGRTTISAGAFS | |
| | | QTHCVTDCQRNEAGLQCDQNGQYRASQKDRGSGKAFCVD | |
| | | GEGRRLPWWETEAPLEDSQCLMMQKFEKVPESKVIFDAN | |
| | | APVAVRSKVPDSEFPVMQCLTDCTEDEACSFFTVSTTEP | |
| | | EISCDFYAWTSDNVACMTSDQKRDALGNSKATSFGSLRC | |
| | | QVKVRSHGQDSPAVYLKKGQGSTTTLQKRFEPTGFQNML | |
| | | SGLYNPIVESASGANLTDAHLFCLLACDRDLCCDGFVLT | |
| | | QVQGGAIICGLLSSPSVLLCNVKDWMDPSEAWANATCPG | |
| | | VTYDQESHQVILRLGDQEFIKSLTPLEGTQDTFTNFQQV | |
| | | YLWKDSDMGSRPESMGCRKNTVPRPASPTEAGLTTELES | |
| | | PVDLNQVIVNGNQSLSSQKHWLFKHLFSAQQANLWCLSR | |
| | | CVQEHSFCQLAEITESASLYFTCTLYPEAQVCDDIMESN | |
| | | AQGCRLILPQMPKALFRKKVILEDKVKNFYTRLPFQKLT | |
| | | GISIRNKVPMSEKSISNGFFECERRCDADPCCTGFGELN | |
| | | VSQLKGGEVTCLTLNSLGIQMCSEENGGAWRILDCGSPD | |
| | | IEVHTYPFGWYQKPIAQNNAPSFCPLVVLPSLTEKVSLD | |
| | | SWQSLALSSVVVDPSIRHEDVAHVSTAATSNFSAVRDLC | |
| | | LSECSQHEACLITTLQTQPGAVRCMFYADTQSCTHSLQG | |
| | | QNCRLLLREEATHIYRKPGISLLSYEASVPSVPISTHGR | |
| | | LLGRSQAIQVGTSWKQVDQFLGVPYAAPPLAERRFQAPE | |
| | | PLNWTGSWDASKPRASCWQPGTRTSTSPGVSEDCLYLNV | |
| | | FIPQNVAPNASVLVFFHNTMDREESEGWPAIDGSFLAAV | |
| | | GNLIVVTASYRVGVFGFLSSGSGEVSGNWGLLDQVAALT | |
| | | WVQTHIRGFGGDPRRVSLAADRGGADVASIHLLTARATN | |
| | | SQLFRRAVLMGGSALSPAAVISHERAQQQAIALAKEVSC | |
| | | PMSSSQEVVSCLRQKPANVLNDAQTKLLAVSGPFHYWGP | |
| | | VIDGHFLREPPARALKRSLWVEVDLLIGSSQDDGLINRA | |
| | | KAVKQFEESQGRTSSKTAFYQALQNSLGGEDSDARVEAA | |
| | | ATWYYSLEHSTDDYASFSRALENATRDYFIICPIIDMAS | |
| | | AWAKRARGNVFMYHAPENYGHGSLELLADVQFALGLPFY | |
| | | PAYEGQFSLEEKSLSLKIMQYFSHFIRSGNPNYPYEFSR | |
| | | KVPTFATPWPDFVPRAGGENYKEFSELLPNRQGLKKADC | |
| | | SFWSKYISSLKTSADGAKGGQSAESEEEELTAGSGLRED | |
| | | LLSLQEPGSKTYSK | |
| Peroxisome | ppara | MVDTESPLCPLSPLEAGDLESPLSEEFLQEMGNIQEISQ | 12 |
| proliferator- | | SIGEDSSGSFGFTEYQYLGSCPGSDGSVITDTLSPASSP | |
| activated receptor | | SSVTYPVVPGSVDESPSGALNIECRICGDKASGYHYGVH | |
| alpha (PPARA) | | ACEGCKGFFRRTIRLKLVYDKCDRSCKIQKKNRNKCQYC | |
| | | RFHKCLSVGMSHNAIRFGRMPRSEKAKLKAEILTCEHDI | |
| | | EDSETADLKSLAKRIYEAYLKNFNMNKVKARVILSGKAS | |
| | | NNPPFVIHDMETLCMAEKTLVAKLVANGIQNKEAEVRIF | |
| | | HCCQCTSVETVTELTEFAKAIPGFANLDLNDQVTLLKYG | |
| | | VYEAIFAMLSSVMNKDGMLVAYGNGFITREFLKSLRKPF | |
| | | CDIMEPKFDFAMKFNALELDDSDISLFVAAIICCGDRPG | |
| | | LLNVGHIEKMQEGIVHVLRLHLQSNHPDDIFLFPKLLQK | |
| | | MADLRQLVTEHAQLVQIIKKTESDAALHPLLQEIYRDMY | |

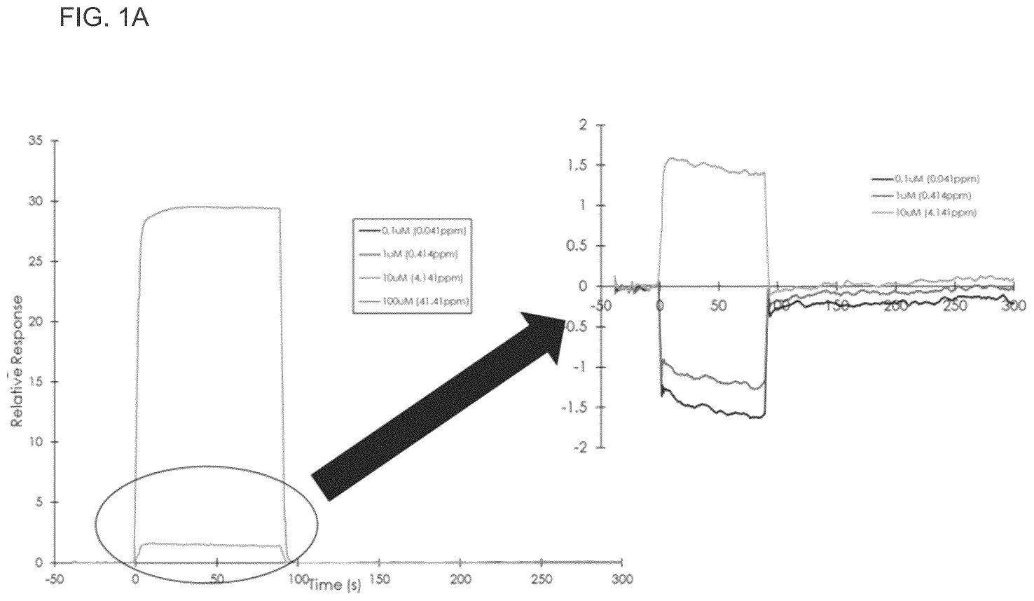

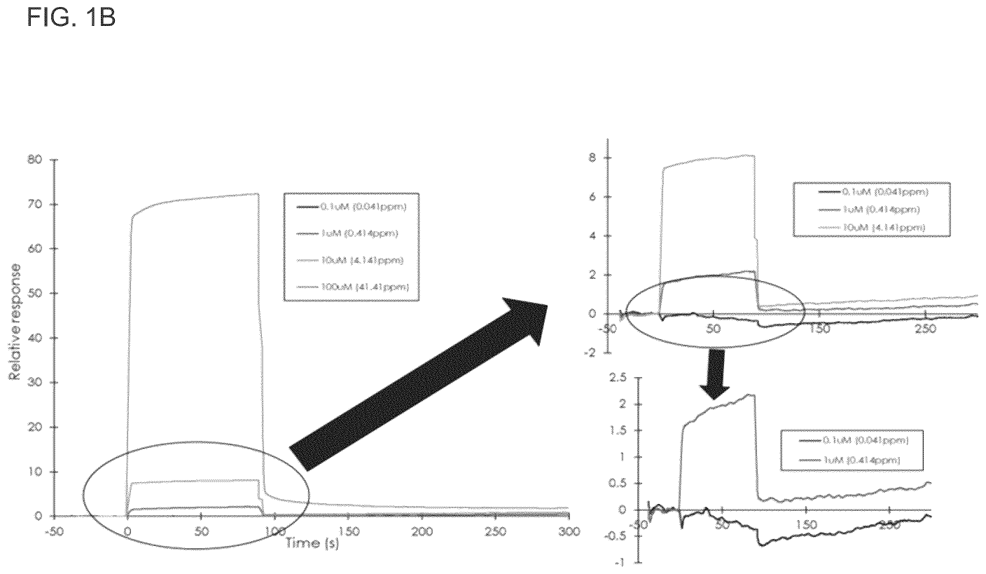

The SPR signal for each of human TG and PPARA are shown in FIGS. 1A and 1B. TG exhibited detectable signal between 41.4 ppm and 4.14 ppm, while PPARA showed detectable signal between 41.4 ppm and 0.414 ppm.

### REFERENCES FOR EXAMPLE 1

(1) Giusto, Paolo, Paola Lova, Giovanni Manfredi, Serena Gazzo, Padmanabhan Srinivasan, Stefano Radice, and Davide Comoretto. “Colorimetric Detection of Perfluorinated Compounds by All-Polymer Photonic Transducers.” ACS Omega 3, no. 7 (Jul. 31, 2018): 7517-22. doi.org/10.1021/acsomega.8b00554.
(2) Park, Junyoung, Kyung-Ae Yang, Yongju Choi, and Jong Kwon Choe. “Novel SsDNA Aptamer-Based Fluorescence Sensor for Perfluorooctanoic Acid Detection in Water.” Environment International 158 (January 2022): 107000. doi.org/10.1016/j.envint.2021.107000.
(3) Cheng, Yu H., Dushyant Barpaga, Jennifer A. Soltis, V. Shutthanandan, Roli Kargupta, Kee Sung Han, B. Peter McGrail, Radha Kishan Motkuri, Sagnik Basuray, and Sayandev Chatterjee. “Metal-Organic Framework-Based Microfluidic Impedance Sensor Platform for Ultrasensitive Detection of Perfluorooctanesulfonate.” ACS Applied Materials & Interfaces 12, no. 9 (Mar. 4, 2020): 10503-14. doi.org/10.1021/acsami.9b22445.
(4) Gallagher A, Kar S, Sepdlveda MS. Computational Modeling of Human Serum Albumin Binding of Per- and Polyfluoroalkyl Substances Employing QSAR, Read-Across, and Docking. Molecules. 2023 Jul. 13; 28(14):5375. doi: 10.3390/molecules28145375. PMID: 37513249; PMCID: PMC10383382.
(5) Chi Q, Li Z, Huang J, Ma J, Wang X. Interactions of perfluorooctanoic acid and perfluorooctanesulfonic acid with serum albumins by native mass spectrometry, fluorescence and molecular docking. Chemosphere. 2018 May; 198:442-449. doi: 10.1016/j.chemosphere.2018.01.152. Epub 2018 Feb. 3. PMID: 29425944.

1.-22. (canceled)

23. A method of detecting a PFAS compound in a sample, comprising:
(a) contacting the sample with a mammalian per- and polyfluoroalkyl compound (PFAS) binding protein, wherein the PFAS binding protein binds to one or more PFAS compounds; and
(b) detecting the level of the mammalian PFAS binding protein-PFAS compound conjugate,
wherein the level of mammalian PFAS binding protein-PFAS compound conjugate is indicative of the level of the PFAS compound in the sample.

24. The method of claim 23, wherein the detecting is by ELISA, an electrochemical method, an interferometry method, a Surface Plasmon Resonance (SPR) method, or a flow-based immunoassay.

25. The method of claim 23, wherein the mammalian PFAS binding protein comprises a mammalian protein selected from the group consisting of Nucleoside diphosphate kinase, mitochondrial (NME4); FAM110D; Tumor protein p63-regulated gene 1 protein (TPRG1); Voltage-gated potassium channel subunit beta-1 (KCNAB1); Protein CutA (CUTA); Ferroptosis suppressor protein 1 (AIFM2); Polymerase delta-interacting protein 3 (POLDIP3); Zinc-binding alcohol dehydrogenase domain-containing protein 2 (ZADH2); Ubiquitin carboxyl-terminal hydrolase 21 (USP21); Lysine-specific demethylase 4D (KDM4D); thyroglobulin; and peroxisome proliferator-activated receptor alpha (PPARA); sequence substantially identical thereto; and a fragment thereof.

26. The method of claim 25, wherein the mammalian PFAS binding protein comprises the sequence set forth in one of SEQ ID NOs: 1-12 or a sequence at least 80% identical thereto.

27. The method of claim 26, wherein the mammalian PFAS binding protein comprises the sequence set forth in one of SEQ ID NOs: 1-12.

28. (canceled)

29. The method of claim 24, wherein the detecting is by a lateral-flow based method comprising lateral flow or competitive lateral flow.

30. The method of claim 23 wherein the limit of detection of the method is no greater than single digit parts per trillion.

31. The method of claim 23, wherein the sample is a water sample.

32. The method of claim 31, comprising pre-treating the water sample by concentrating the PFAS compound before step (a).

33. The method of claim 32, comprising pre-treating the water sample by removing inhibitors of the binding of the mammalian PFAS binding protein and the PFAS compound before step (a).

34. A method of detecting a PFAS compound in a sample, comprising:
(a) contacting the sample with a mammalian per- and polyfluoroalkyl compound (PFAS) binding protein, wherein the PFAS binding protein binds to one or more PFAS compounds, wherein the mammalian PFAS binding protein is bound to an electroactive surface to create a mammalian PFAS binding protein-bound base, and wherein the contacting is under conditions that allow the PFAS binding protein to bind the PFAS compound to result in a mammalian PFAS binding protein-PFAS compound conjugate;
(b) measuring electrochemical signals with a transducer before and after binding of the PFAS compound to the mammalian PFAS binding protein-bound base and calculating a difference between the electrochemical signals,
wherein a difference between the electrochemical signals before and after binding of the PFAS compound to the mammalian PFAS binding protein-bound base correlates to the amount of the PFAS compound in the sample.

35. The method of claim 34, wherein the sample is a water sample.

36. The method of claim 34, wherein the mammalian PFAS binding protein comprises a mammalian protein selected from the group consisting of Nucleoside diphosphate kinase, mitochondrial (NME4); FAM110D; Tumor protein p63-regulated gene 1 protein (TPRG1); Voltage-gated potassium channel subunit beta-1 (KCNAB1); Protein CutA (CUTA); Ferroptosis suppressor protein 1 (AIFM2); Polymerase delta-interacting protein 3 (POLDIP3); Zinc-binding alcohol dehydrogenase domain-containing protein 2 (ZADH2); Ubiquitin carboxyl-terminal hydrolase 21 (USP21); Lysine-specific demethylase 4D (KDM4D); thyroglobulin; and peroxisome proliferator-activated receptor alpha (PPARA); sequence substantially identical thereto; and a fragment thereof.

37. The method of claim 36, wherein the mammalian PFAS binding protein comprises the sequence set forth in one of SEQ ID NOs: 1-12 or a sequence at least 80% identical thereto.

38. The method of claim 37, wherein the mammalian PFAS binding protein comprises the sequence set forth in one of SEQ ID NOs: 1-12.

39.-42. (canceled)

43. A method of detecting a PFAS compound in a water sample, comprising:
(a) contacting the sample to a solid substrate, wherein a mammalian per- and polyfluoroalkyl compound (PFAS) binding protein that binds to one or more PFAS compounds is immobilized on the solid substrate and is attached to a detectable label, under conditions suitable for the PFAS compound to bind to the mammalian PFAS binding protein to generate a PFAS compound-mammalian PFAS binding protein conjugate; wherein the solid substrate comprises a first end, a first middle portion, a second middle portion, and a second end, wherein the solid substrate allows liquid to migrate from the first end through the middle portions to the second end through capillary action; wherein the first middle portion comprises the mammalian PFAS binding protein and the second middle portion comprises (i) a second binding molecule immobilized on the solid substrate, wherein the second binding molecule binds to the PFAS compound-mammalian PFAS binding protein conjugate, or ii) the PFAS binding protein immobilized on the solid substrate; and wherein the sample is contacted to the first end of the solid substrate;
(b) allowing the PFAS compound-mammalian PFAS binding protein conjugate to migrate from the first end through first middle portion into the second middle portion; and,
(c) if the second middle portion comprises a second binding molecule immobilized on the solid substrate, detecting the detectable label in the second middle portion, wherein detecting the detectable label indicates the presence of the PFAS compound in the sample; or if the second middle portion comprises the PFAS binding protein immobilized on the solid substrate, detecting the detectable label in the second middle portion, wherein the absence of the detectable label indicates the presence of the PFAS compound in the sample.

44. The method of claim 43, wherein the detectable signal is colorimetric or fluorescent.

45. The method of claim 43, wherein the mammalian PFAS binding protein comprises a mammalian protein selected from the group consisting of Nucleoside diphosphate kinase, mitochondrial (NME4); FAM110D; Tumor protein p63-regulated gene 1 protein (TPRG1); Voltage-gated potassium channel subunit beta-1 (KCNAB1); Protein CutA (CUTA); Ferroptosis suppressor protein 1 (AIFM2); Polymerase delta-interacting protein 3 (POLDIP3); Zinc-binding alcohol dehydrogenase domain-containing protein 2 (ZADH2); Ubiquitin carboxyl-terminal hydrolase 21 (USP21); Lysine-specific demethylase 4D (KDM4D); thyroglobulin; and peroxisome proliferator-activated receptor alpha (PPARA); sequence substantially identical thereto; and a fragment thereof.

46. The method of claim 45, wherein the mammalian PFAS binding protein comprises the sequence set forth in one of SEQ ID NOs: 1-12 or a sequence at least 80% identical thereto.

47. The method of claim 46, wherein mammalian PFAS binding protein comprises the sequence set forth in one of SEQ ID NOs: 1-12.


SEQUENCE LISTING (12 sequences)

SEQ ID NO: 1 - protein, 187 aa, Homo sapiens
MGGLFWRSALRGLRCGPRAPGPSLLVRHGSGGPSWTRERTLVAVKPDGVQRRLVGDVIQRFERRGFTLVGMKMLQAPESVLAEHYQDLRRKPFYPALIRYMSSGPVVAMVWEGYNVVRASRAMIGHTDSAEAAPGTIRGDFSVHISRNVIHASDSVEGAQREIQLWFQSSELVSWADGGQHSSIHPA

SEQ ID NO: 2 - protein, 271 aa, Homo sapiens
MLLAPPSTPSRGRTPSAVERLEADKAKYVKTHQVIARRQEPALRGSPGPLTPHPCNELGPPASPRTPRPVRRGSGRRLPRPDSLIFYRQKRDCKASVNKENAKGQGLVRRLFLGAPRDAAPSSPASTERPAASGGWAAPQDAPEAAGKRALCPTCSLPLSEKERFFNYCGLERALVEVLGAERFSPQSWGADASPQAGTSPPPGSGDASDWTSSDRGVDSPGGAGGGGGSEAAGSARDRRPPVSVVERNARVIQWLYGCQRARGPPRESEV

SEQ ID NO: 3 - protein, 275 aa, Homo sapiens
MSTIGSFEGFQAVSLKQEGDDQPSETDHLSMEEEDPMPRQISRQSSVTESTLYPNPYHQPYISRKYFATRPGAIETAMEDLKGHVAETSGETIQGFWLLTKIDHWNNEKERILLVTDKTLLICKYDFIMLSCVQLQRIPLSAVYRICLGKFTFPGMSLDKRQGEGLRIYWGSPEEQSLLSRWNPWSTEVPYATFTEHPMKYTSEKFLEICKLSGFMSKLVPAIQNAHKNSTGSGRGKKLMVLTEPILIETYTGLMSFIGNRNKLGYSLARGSIGF

SEQ ID NO: 4 - protein, 419 aa, Homo sapiens
MLAARTGAAGSQISEENTKLRRQSGFSVAGKDKSPKKASENAKDSSLSPSGESQLRARQLALLREVEMNWYLKLCDLSSEHTTVCTTGMPHRNLGKSGLRVSCLGLGTWVTFGGQISDEVAERLMTIAYESGVNLFDTAEVYAAGKAEVILGSIIKKKGWRRSSLVITTKLYWGGKAETERGLSRKHIIEGLKGSLQRLQLEYVDVVFANRPDSNTPMEEIVRAMTHVINQGMAMYWGTSRWSAMEIMEAYSVARQFNMIPPVCEQAEYHLFQREKVEVQLPELYHKIGVGAMTWSPLACGIISGKYGNGVPESSRASLKCYQWLKERIVSEEGRKQQNKLKDLSPIAERLGCTLPQLAVAWCLRNEGVSSVLLGSSTPEQLIENLGAIQVLPKMTSHVVNEIDNILRNKPYSKKDYRS

SEQ ID NO: 5 - protein, 179 aa, Homo sapiens
MSGGRAPAVLLGGVASLLLSFVWMPALLPVASRLLLLPRVLLTMASGSPPTQPSPASDSGSGYVPGSVSAAFVTCPNEKVAKEIARAVVEKRLAACVNLIPQITSIYEWKGKIEEDSEVLMMIKTQSSLVPALTDFVRSVHPYEVAEVIALPVEQGNFPYLQWVRQVTESVSDSITVLP

SEQ ID NO: 6 - protein, 373 aa, Homo sapiens
MGSQVSVESGALHVVIVGGGFGGIAAASQLQALNVPFMLVDMKDSFHHNVAALRASVETGFAKKTFISYSVTFKDNFRQGLVVGIDLKNQMVLLQGGEALPFSHLILATGSTGPFPGKFNEVSSQQAAIQAYEDMVRQVQRSRFIVVVGGGSAGVEMAAEIKTEYPEKEVTLIHSQVALADKELLPSVRQEVKEILLRKGVQLLLSERVSNLEELPLNEYREYIKVQTDKGTEVATNLVILCTGIKINSSAYRKAFESRLASSGALRVNEHLQVEGHSNVYAIGDCADVRTPKMAYLAGLHANIAVANIVNSVKQRPLQAYKPGALTFLLSMGRNDGVGQISGFYVGRLMVRLTKSRDLFVSTSWKTMRQSPP

SEQ ID NO: 7 - protein, 421 aa, Homo sapiens
MADISLDELIRKRGAAAKGRLNARPGVGGVRSRVGIQQGLLSQSTRTATFQQRFDARQKIGLSDARLKLGVKDAREKLLQKDARFRIKGKVQDAREMLNSRKQQTTVPQKPRQVADAREKISLKRSSPAAFINPPIGTVTPALKLTKTIQVPQQKAMAPLHPHPAGMRINVVNNHQAKQNLYDLDEDDDGIASVPTKQMKFAASGGFLHHMAGLSSSKLSMSKALPLTKVVQNDAYTAPALPSSIRTKALTNMSRTLVNKEEPPKELPAAEPVLSPLEGTKMTVNNLHPRVTEEDIVELFCVCGALKRARLVHPGVAEVVFVKKDDAITAYKKYNNRCLDGQPMKCNLHMNGNVITSDQPILLRLSDSPSMKKESELPRRVNSASSSNPPAEVDPDTILKALFKSSGASVTTQPTEFKIKL

SEQ ID NO: 8 - protein, 377 aa, Homo sapiens
MLRLVPTGARAIVDMSYARHFLDFQGSAIPQAMQKLVVTRLSPNFREAVTLSRDCPVPLPGDGDLLVRNRFVGVNASDINYSAGRYDPSVKPPFDIGFEGIGEVVALGLSASARYTVGQAVAYMAPGSFAEYTVVPASIATPVPSVKPEYLTLLVSGTTAYISLKELGGLSEGKKVLVTAAAGGTGQFAMQLSKKAKCHVIGTCSSDEKSAFLKSLGCDRPINYKTEPVGTVLKQEYPEGVDVVYESVGGAMFDLAVDALATKGRLIVIGFISGYQTPTGLSPVKAGTLPAKLLKKSASVQGFFLNHYLSKYQAAMSHLLEMCVSGDLVCEVDLGDLSPEGRFTGLESIFRAVNYMYMGKNTGKIVVELPHSVNSKL

SEQ ID NO: 9 - protein, 565 aa, Homo sapiens
MPQASEHRLGRTREPPVNIQPRVGSKLPFAPRARSKERRNPASGPNPMLRPLPPRPGLPDERLKKLELGRGRTSGPRPRGPLRADHGVPLPGSPPPTVALPLPSRTNLARSKSVSSGDLRPMGIALGGHRGTGELGAALSRLALRPEPPTLRRSTSLRRLGGFPGPPTLFSIRTEPPASHGSFHMISARSSEPFYSDDKMAHHTLLLGSGHVGLRNLGNTCFLNAVLQCLSSTRPLRDFCLRRDFRQEVPGGGRAQELTEAFADVIGALWHPDSCEAVNPTRFRAVFQKYVPSFSGYSQQDAQEFLKLLMERLHLEINRRGRRAPPILANGPVPSPPRRGGALLEEPELSDDDRANLMWKRYLEREDSKIVDLFVGQLKSCLKCQACGYRSTTFEVFCDLSLPIPKKGFAGGKVSLRDCFNLFTKEEELESENAPVCDRCRQKTRSTKKLTVQRFPRILVLHLNRFSASRGSIKKSSVGVDFPLQRLSLGDFASDKAGSPVYQLYALCNHSGSVHYGHYTALCRCQTGWHVYNDSRVSPVSENQVASSEGYVLFYQLMQEPPRCL

SEQ ID NO: 10 - protein, 523 aa, Homo sapiens
METMKSKANCAQNPNCNIMIFHPTKEEFNDFDKYIAYMESQGAHRAGLAKIIPPKEWKARETYDNISEILIATPLQQVASGRAGVFTQYHKKKKAMTVGEYRHLANSKKYQTPPHQNFEDLERKYWKNRIYNSPIYGADISGSLFDENTKQWNLGHLGTIQDLLEKECGVVIEGVNTPYLYFGMWKTTFAWHTEDMDLYSINYLHLGEPKTWYVVPPEHGQRLERLARELFPGSSRGCGAFLRHKVALISPTVLKENGIPFNRITQEAGEFMVTFPYGYHAGFNHGFNCAEAINFATPRWIDYGKMASQCSCGEARVTFSMDAFVRILQPERYDLWKRGQDRAVVDHMEPRVPASQELSTQKEVQLPRRAALGLRQLPSHWARHSPWPMAARSGTRCHTLVCSSLPRRSAVSGTATQPRAAAVHSSKKPSSTPSSTPGPSAQIIHPSNGRRGRGRPPQKLRAQELTLQTPAKRPLLAGTTCTASGPEPEPLPEDGALMDKPVPLSPGLQHPVKASGCSWAPVP

SEQ ID NO: 11 - protein, 1574 aa, Homo sapiens
IPRKPISKRPVRPSLPRSPRCPLPFNASEVVGGTILCETISGPTGSAMQQCQLLCRQGSWSVFPPGPLICSLESGRWESQLPQPRACQRPQLWQTIQTQGHFQLQLPPGKMCSADYAGLLQTFQVFILDELTARGFCQIQVKTFGTLVSIPVCNNSSVQVGCLTRERLGVNVTWKSRLEDIPVASLPDLHDIERALVGKDLLGRFTDLIQSGSFQLHLDSKTFPAETIRFLQGDHFGTSPRTWFGCSEGFYQVLTSEASQDGLGCVKCPEGSYSQDEECIPCPVGFYQEQAGSLACVPCPVGRTTISAGAFSQTHCVTDCQRNEAGLQCDQNGQYRASQKDRGSGKAFCVDGEGRRLPWWETEAPLEDSQCLMMQKFEKVPESKVIFDANAPVAVRSKVPDSEFPVMQCLTDCTEDEACSFFTVSTTEPEISCDFYAWTSDNVACMTSDQKRDALGNSKATSFGSLRCQVKVRSHGQDSPAVYLKKGQGSTTTLQKRFEPTGFQNMLSGLYNPIVFSASGANLTDAHLFCLLACDRDLCCDGFVLTQVQGGAIICGLLSSPSVLLCNVKDWMDPSEAWANATCPGVTYDQESHQVILRLGDQEFIKSLTPLEGTQDTFTNFQQVYLWKDSDMGSRPESMGCRKNTVPRPASPTEAGLTTELFSPVDLNQVIVNGNQSLSSQKHWLFKHLFSAQQANLWCLSRCVQEHSFCQLAEITESASLYFTCTLYPEAQVCDDIMESNAQGCRLILPQMPKALFRKKVILEDKVKNFYTRLPFQKLTGISIRNKVPMSEKSISNGFFECERRCDADPCCTGFGFLNVSQLKGGEVTCLTLNSLGIQMCSEENGGAWRILDCGSPDIEVHTYPFGWYQKPIAQNNAPSFCPLVVLPSLTEKVSLDSWQSLALSSVVVDPSIRHFDVAHVSTAATSNFSAVRDLCLSECSQHEACLITTLQTQPGAVRCMFYADTQSCTHSLQGQNCRLLLREEATHIYRKPGISLLSYEASVPSVPISTHGRLLGRSQAIQVGTSWKQVDQFLGVPYAAPPLAERRFQAPEPLNWTGSWDASKPRASCWQPGTRTSTSPGVSEDCLYLNVFIPQNVAPNASVLVFFHNTMDREESEGWPAIDGSFLAAVGNLIVVTASYRVGVFGFLSSGSGEVSGNWGLLDQVAALTWVQTHIRGFGGDPRRVSLAADRGGADVASIHLLTARATNSQLFRRAVLMGGSALSPAAVISHERAQQQAIALAKEVSCPMSSSQEVVSCLRQKPANVLNDAQTKLLAVSGPFHYWGPVIDGHFLREPPARALKRSLWVEVDLLIGSSQDDGLINRAKAVKQFEESQGRTSSKTAFYQALQNSLGGEDSDARVEAAATWYYSLEHSTDDYASFSRALENATRDYFIICPIIDMASAWAKRARGNVFMYHAPENYGHGSLELLADVQFALGLPFYPAYEGQFSLEEKSLSLKIMQYFSHFIRSGNPNYPYEFSRKVPTFATPWPDFVPRAGGENYKEFSELLPNRQGLKKADCSFWSKYISSLKTSADGAKGGQSAESEEEELTAGSGLREDLLSLQEPGSKTYSK

SEQ ID NO: 12 - protein, 468 aa, Homo sapiens
MVDTESPLCPLSPLEAGDLESPLSEEFLQEMGNIQEISQSIGEDSSGSFGFTEYQYLGSCPGSDGSVITDTLSPASSPSSVTYPVVPGSVDESPSGALNIECRICGDKASGYHYGVHACEGCKGFFRRTIRLKLVYDKCDRSCKIQKKNRNKCQYCRFHKCLSVGMSHNAIRFGRMPRSEKAKLKAEILTCEHDIEDSETADLKSLAKRIYEAYLKNFNMNKVKARVILSGKASNNPPFVIHDMETLCMAEKTLVAKLVANGIQNKEAEVRIFHCCQCTSVETVTELTEFAKAIPGFANLDLNDQVTLLKYGVYEAIFAMLSSVMNKDGMLVAYGNGFITREFLKSLRKPFCDIMEPKFDFAMKFNALELDDSDISLFVAAIICCGDRPGLLNVGHIEKMQEGIVHVLRLHLQSNHPDDIFLFPKLLQKMADLRQLVTEHAQLVQIIKKTESDAALHPLLQEIYRDMY